(19) United States
(12) Patent Application Publication
(10) Pub. No.: US 2026/0232492 A1
(43) Pub. Date: Aug. 13, 2026

(54) WOUND DRESSING

(71) Applicant: TAICEND TECHNOLOGY CO., LTD., Kachsiung City (TW)

(72) Inventors: Yu-Pu Chu, Kaohsiung (TW); Fu-Chuan Chang, Kaohsiung City (TW); Tsz-Yi Tang, Kaohsiung City (TW)

(21) Appl. No.: 19/049,851

(22) Filed: Feb. 10, 2025

(51) Int. Cl. A61F 13/00

(52) U.S. Cl. CPC A61F 13/00; A61F 2013/00255

(57) ABSTRACT

A wound dressing includes a main body, a positioning member, and a retaining member. The main body has a fastening end portion, a connecting end portion, and a through hole disposed between the fastening end portion and the connecting end portion. The positioning member is integrally connected to the connecting end portion and is configured to be inserted through the through hole such that the main body forms into a ring shape. The retaining member is connected to the fastening end portion and is configured to be fastened in proximity to the connecting end portion when the positioning member is inserted through the through hole so as to keep the main body in the ring shape.

### FIELD

The disclosure relates to a wound dressing.

### BACKGROUND

A circumcision operation is a common surgical operation. After the circumcision operation, the wound is commonly dressed with non-elastic gauze, and no specialized dressings are available to treat the wound. Furthermore, non-elastic gauze may not be satisfactory for applications on wounds on penises. First, penises can vary in size from patient to patient and the size of a patient's penis can change depending on external environmental factors or physiological factors. Furthermore, wound drainage may occur. Thus, there exist needs to effectively absorb the drainage (tissue fluid) from the wound to keep the wound dry, to minimize patient discomfort, to reduce frequency of dressing replacements, and to facilitate postoperative healing.

### SUMMARY

Therefore, an object of the disclosure is to provide a wound dressing that can alleviate at least one of the drawbacks of the prior art.

According to the disclosure, the wound dressing includes a main body, a positioning member, and a retaining member.

The main body is elongated along a lengthwise direction, is elastic and absorbent, and has a fastening end portion, a connecting end portion, and a through hole. The connecting end portion is opposite to the fastening end portion in the lengthwise direction. The through hole is disposed between the fastening end portion and the connecting end portion.

The positioning member is integrally connected to the connecting end portion. The positioning member is configured to be inserted through the through hole such that the main body forms into a ring shape.

The retaining member is connected to the fastening end portion. The retaining member is configured to be fastened in proximity to the connecting end portion when the positioning member is inserted through the through hole so as to keep the main body in the ring shape.

### BRIEF DESCRIPTION OF THE DRAWINGS

Other features and advantages of the disclosure will become apparent in the following detailed description of the embodiments with reference to the accompanying drawings. It is noted that features may not be drawn to scale.

FIG. 1 is a perspective view illustrating a wound dressing according to a first embodiment of the disclosure.

FIG. 2 is a side view of the first embodiment

FIG. 3 is a schematic view of the first embodiment illustrating how to use the wound dressing.

FIG. 4 is a perspective view illustrating a wound dressing according to a second embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

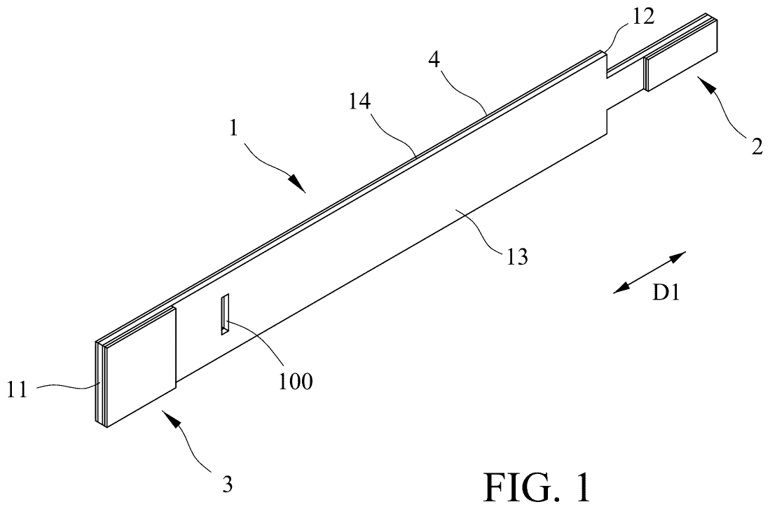

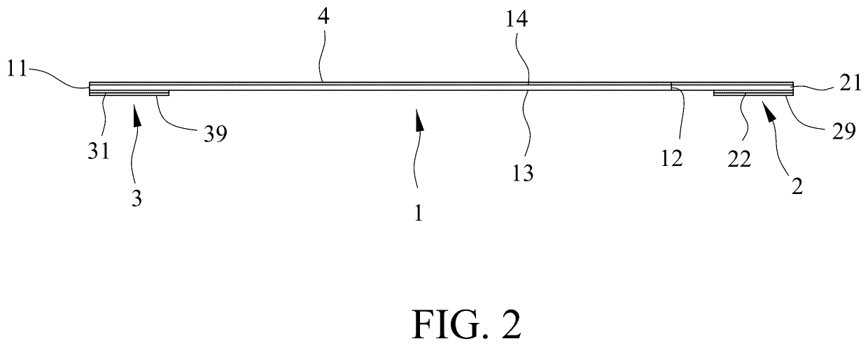

FIGS. 1 and 2 illustrate a wound dressing according to a first embodiment of the disclosure. The wound dressing includes a main body 1, a positioning member 2, a retaining member 3, and a waterproof film 4. The wound dressing of the disclosure may serve as a circumcision dressing in this embodiment. In other embodiments, the wound dressing of the disclosure may be dressed on injuries not caused by the circumcision operation.

The main body 1 is elongated along a lengthwise direction (D1) and is elastic and absorbent. The main body 1 is made of a hydrophilic foaming material that is composed of polyethylene glycol polyurethane. The main body 1 has a plurality of pores (not shown in the drawings) each of which has a pore size ranging from 400 μm to 800 μm, thereby optimizing liquid absorbency. In addition, the main body 1 has a thickness ranging from 0.5 mm to 3.5 mm in a transverse direction perpendicular to the lengthwise direction (D1), thereby facilitating convenience for dressing a wound and maintaining elasticity of the main body 1. In this embodiment, the main body 1 has a fastening end portion 11, a connecting end portion 12, an inner surface 13, an outer surface 14, and a through hole 100. The connecting end portion 12 is opposite to the fastening end portion 11 in the lengthwise direction (D1). The inner surface 13 and the outer surface 14 are opposite to each other and extend between the fastening end portion 11 and the connecting end portion 12. The inner surface 13 is suitable for facing a wound region. The through hole 100 extends through the inner surface 13 and the outer surface 14 and is spaced apart from the fastening end portion 11. A distance between the fastening end portion 11 and the through hole 100 in the lengthwise direction (D1) is smaller than a distance between the through hole 100 and the connecting end portion 12 in the lengthwise direction (D1).

The positioning member 2 is integrally connected to the connecting end portion 12 of the main body 1. The positioning member 2 is configured to be inserted through the through hole 100 of the main body 1 such that the main body 1 forms into a ring shape. As shown in FIG. 1, in a width direction perpendicular to the lengthwise direction (D1) and a thickness direction, a width of the positioning member 2 is smaller than a width of the connecting end portion 12. The positioning member 2 has a strap portion 21 and a gluey portion 22 disposed on the strap portion 21. The strap portion 21 is configured to be inserted through the through hole 100. The gluey portion 22 is disposed on the strap portion 21. The gluey portion 22 is configured to be glued to the main body 1 when the positioning member 2 is inserted through the through hole 100. When the wound dressing of the disclosure is not in use, the gluey portion 22 may be covered by a release paper 29 so as to maintain the adhesiveness of the gluey portion 22.

The retaining member 3 is connected to the fastening end portion 11 of the main body 1. The retaining member 3 is configured to be fastened to the outer surface 14 in proximity to the connecting end portion 12 when the positioning member 2 is inserted through the through hole 100 so as to keep the main body 1 in the ring shape. In this embodiment, the retaining member 3 is disposed on the inner surface 13 and has an adhesive layer 31. The adhesive layer 31 and the inner surface 13 are oriented in a same direction. When the wound dressing of the disclosure is not in use, the adhesive layer 31 may be covered by a release paper 39 so as to maintain the adhesiveness of the adhesive layer 31.

The waterproof film 4 is formed on the outer surface 14 of the main body 1. The waterproof film 4 is made of a polyurethane material so as to prevent the outer surface 14 from absorbing fluids. By virtue of the waterproof film 4, the liquid absorbency of the inner surface 13 is ensured and the outer surface 14 is waterproof. It should be noted that the waterproof film 4 formed on the outer surface 14 of the main body 1 may enhance the overall strength of the wound dressing. As a result, the elasticity and stretchability of the wound dressing may be enhanced and the wound dressing may better facilitate wound care. The waterproof film 4 may compensate for the deficiency in the stretchability of the main body 1.

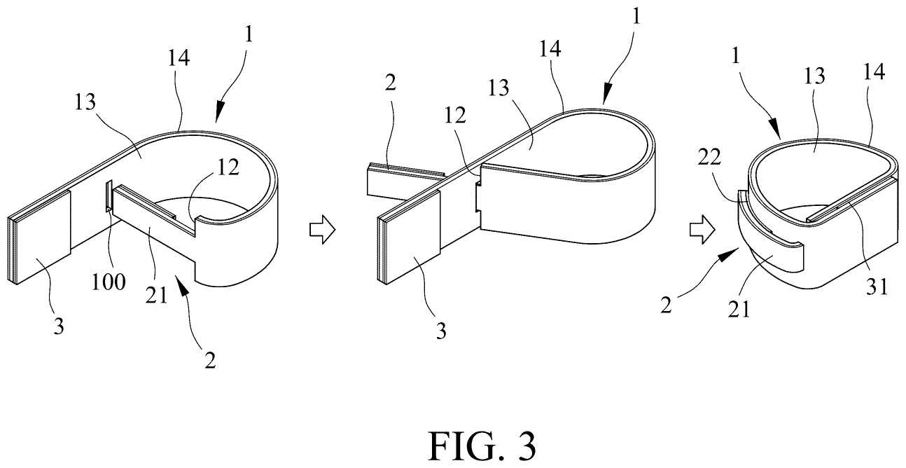

Referring to FIG. 3 in combination with FIG. 1, by virtue of the thickness and elasticity of the main body 1, the main body 1 may be easily bent to direct the positioning member 2 toward the through hole 100. After the strap portion 21 is inserted through the through hole 100, the main body 1 is formed into the ring shape. By varying the extent to which the positioning member 2 is inserted through the through hole 100, the ring formed by the main body 1 is adjustable in size to fit the size of the wound region of the patient. The elasticity of the main body 1 prevents the main body 1 in the ring state from curling. After the ring formed by the main body is adjusted in size, the gluey portion 22 of the positioning member 2 is attached to the outer surface 14 of the main body 1 so that the positioning member 2 is secured to the main body 1. When the adhesive layer 31 of the retaining member 3 is attached to the outer surface 14 in proximity to the connecting end portion 12, the main body 1 is kept in the ring shape, and the application of the wound dressing to the wound region is complete.

After the wound dressing of the disclosure is applied to the wound region of the patient, because the main body 1 is elastic, and because the retaining member 3 attached to the outer surface 14 stably keeps the main body 1 in the ring shape, the main body 1 may be stably attached to the wound region, may reduce pressure on the wound region, and may also reduce effects of external forces on the wound region. Furthermore, the wound dressing of the disclosure may protect the wound region from chafing. The waterproof film 4 shields the wound region from external fluids, thereby resisting against pathogen intrusion caused by the external fluids and preventing the wound dressing from becoming ineffective due to moisture caused by the external fluids. Because the wound region is directly in contact with the inner surface 13 of the main body 1, the inner surface 13 is able to absorb the tissue fluid from the wound region. Because the main body 1 is made of the hydrophilic foaming material that is composed of polyethylene glycol polyurethane, the tissue fluid absorbed by the main body 1 is prevented from overflowing outwardly. In this embodiment, the pores of the main body 1 are blind pores. The absorbed tissue fluid is locked in the pores of the main body 1, thereby preventing the absorbed tissue fluid from seeping back to the wound region and preventing new tissues from growing within the pores of the main body 1. Thus, the inner surface 13 of the main body 1 is wet but will not stick to the wound region. As a result, patient discomfort from the application of the wound dressing to the wound region may be minimized and the wound dressing may facilitate recovery of the patient.

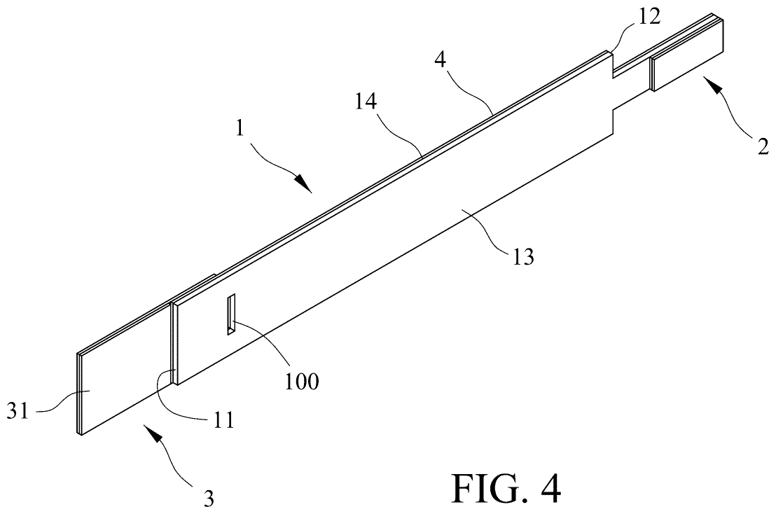

FIG. 4 illustrates a wound dressing according to a second embodiment of the disclosure, which has a structure generally similar to that of the first embodiment. However, in the second embodiment, the retaining member 3 extends outwardly from the fastening end portion 11 and extends away from the connecting end portion 12. In this embodiment, the retaining member 3 is not integrally connected to the main body 1. After the adjustment is made to the size of the main body 1 that is formed into the ring shape, the adjusted size of the main body 1 is not adversely affected by removing the release paper 39 (see FIG. 2) from the retaining member 3 or attaching the retaining member 3 to the outer surface 14 of the main body 1. The wound dressing of the second embodiment achieves the same effects as those of the first embodiment.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A wound dressing comprising:
a main body that is elongated along a lengthwise direction, that is elastic and absorbent, and that has
a fastening end portion,
a connecting end portion opposite to said fastening end portion in the lengthwise direction, and
a through hole disposed between said fastening end portion and said connecting end portion;
a positioning member that is integrally connected to said connecting end portion, said positioning member being configured to be inserted through said through hole such that said main body forms into a ring shape; and
a retaining member that is connected to said fastening end portion, said retaining member being configured to be fastened in proximity to said connecting end portion when said positioning member is inserted through said through hole so as to keep said main body in the ring shape.

2. The wound dressing as claimed in claim 1, wherein said main body is made of an hydrophilic foaming material that is composed of polyethylene glycol polyurethane.

3. The wound dressing as claimed in claim 2, wherein said main body has a thickness ranging from 0.5 mm to 3.5 mm in a transverse direction perpendicular to said lengthwise direction.

4. The wound dressing as claimed in claim 2, wherein said main body further has a plurality of pores each of which has a pore size ranging from 400 μm to 800 μm.

5. The wound dressing as claimed in claim 1, wherein said through hole of said main body is spaced apart from said fastening end portion, a distance between said fastening end portion and said through hole in the lengthwise direction being smaller than a distance between said through hole and said connecting end portion in the lengthwise direction.

6. The wound dressing as claimed in claim 1, wherein:
said main body further has an inner surface and an outer surface opposite to each other and both extending between said fastening end portion and said connecting end portion, said through hole extending through said inner surface and said outer surface; and
said retaining member has an adhesive layer, said adhesive layer and said inner surface being oriented in a same direction.

7. The wound dressing as claimed in claim 6, wherein said retaining member is disposed on said inner surface.

8. The wound dressing as claimed in claim 6, wherein said retaining member extends outwardly from said fastening end portion and extends away from said connecting end portion.

9. The wound dressing as claimed in claim 2, wherein:
said main body further has an inner surface and an outer surface opposite to each other and both extending between said fastening end portion and said connecting end portion; and
said wound dressing further comprises a waterproof film formed on said outer surface.

10. The wound dressing as claimed in claim 9, wherein said waterproof film is made of a polyurethane material.

11. The wound dressing as claimed in claim 1, wherein said positioning member has a strap portion and a gluey portion disposed on said strap portion, said gluey portion being configured to be glued to said main body when said positioning member is inserted through said through hole.
